(19) United States
(12) Patent Application Publication
(10) Pub. No.: US 2023/0240964 A1
(43) Pub. Date: Aug. 3, 2023

(54) SKIN-FIRMING COSMETIC COMPOSITIONS

(71) Applicant: Elastintech, Inc., Greenville, SC (US)

(72) Inventor: Naren VYAVAHARE, Simpsonville, SC (US)

(21) Appl. No.: 18/089,324

(22) Filed: Dec. 27, 2022

Related U.S. Application Data
(60) Provisional application No. 63/294,004, filed on Dec. 27, 2021.

(51) Int. Cl. A61K 8/60; A61K 8/73; A61K 8/44; A61K 8/06; A61Q 19/08; A61Q 19/00

(52) U.S. Cl. CPC A61K 8/602; A61K 8/735; A61K 8/44; A61K 8/062; A61Q 19/08; A61Q 19/007

(57) ABSTRACT

Cosmetic compositions comprising a cosmetic carrier, glucosyl pentagallate, one or more hyaluronic acid, and optionally aminobutyric acid provide improved skin firmness, appearance, and reduced fine lines associated with the appearance of aged skin are disclosed.

### CROSS-REFERENCE TO RELATED APPLICATIONS

This application claims the benefit of and priority to U.S. Provisional Application No. 63/294,004, filed Dec. 27, 2021, which is incorporated herein by reference in its entirety.

### BACKGROUND

Aging and exposure to environmental factors such as UV exposure from the sun can cause undesirable changes in the appearance and physiology of the skin, for example the development of fine lines and wrinkles, loss of elasticity, increased sagging, and loss of firmness in the skin. Many cosmetic products propose to tighten and firm the skin and/or reduce wrinkles, but generally such products achieve this affect by acting as effective moisturizers which temporarily reduce the loss of water from the skin and “plump” the skin, thereby making fine lines and wrinkles appear less noticeable. However, the effects of such products are minimal and temporary, and do not measurably change the elasticity or firmness of the skin.

Products containing retinol can thicken the skin by inhibiting collagen breakdown and increasing the rate of replacement of skin cells. However, such products typically require daily application for 3-6 months before noticeable differences in the skin are apparent. In addition, retinol products can cause skin irritation and can sensitize the skin to sun exposure.

There is therefore a need for new products that can be easily applied to the skin, do not cause skin irritation or sun sensitivity, and provide more long-lasting improvements in skin firmness and elasticity. The cosmetic products of the present disclosure provide such improved properties.

### SUMMARY OF THE INVENTION

In various embodiments, the present disclosure is directed to topical compositions, particularly topical cosmetic compositions, comprising about 0.2-0.6 wt. % glucosyl pentagallate, about 0.02-0.07 wt. % micro HA, and about 0.05-0.15 wt. % mini HA, in combination with one or more cosmetically acceptable excipients selected from the group consisting of a UV protectant, an emollient, a moisturizer, an antibacterial agent, and an emulsifier.

In other embodiments, the compositions of the present disclosure further comprise about 0.02-0.07 wt. % aminobutyric acid.

In various embodiments, the various compositions of the present disclosure described herein after daily administration of 1-10 g/cm² on the epidermis of a subject for at least 28 days, the epidermis has improved firmness, skin thickness, elasticity, increased hydration, reduced roughness, or reduced wrinkle depth.

The compositions of the present disclosure can have any form suitable for cosmetic applications, including serums, creams, emulsions, etc.

In particular embodiments of the compositions of the present disclosure, such compositions comprise about 0.5 wt. % glucosyl pentagallate, about 0.05 wt. % aminobutyric acid, about 0.05 wt. % micro HA, and about 0.10 wt. % mini HA.

### DESCRIPTION OF THE FIGURES

FIG. 1A: absolute insoluble elastin concentration after 14 days; FIG. 1B: insoluble elastin as function of total protein after 14 days.

FIG. 2: Average skin roughness after treatment with neck firming cream of Example 1, prior to (D0), and after 28 (D28) and 56 (D56) days of application.

FIG. 3: Percentage of volunteers showing average roughness improvement after 28 days (D28) and 56 days (D56) of application of neck firming composition of Example 1.

FIG. 4: Firmness improvement compared to control (vehicle only) after 28 days (D28) and 56 days (D56) of application of neck firming composition of Example 1.

FIG. 5: Ultrascan images of representative skin samples taken prior to (D0), and after 28 (D28) and 56 (D56) days of application of the neck firming composition of Example 1.

FIG. 6: High resolution images showing improved skin appearance, taken prior to (D0), and after 28 (D28) and 56 (D56) days of application of the neck firming composition of Example 1.

FIG. 7: Wrinkle depth images showing reduced depth of wrinkles in a representative subject, taken prior to (D0), and after 28 (D28) and 56 (D56) days of application of the neck firming composition of Example 1.

FIG. 8: Subjective perceptions of subjects after use of neck firming composition of Example 1 at 28 (D28) and 56 (D56) days.

FIG. 9: Average skin roughness initially (D0), and after 28 (D28) and 56 (D56) days of application of the eye cream of Example 2.

FIG. 10: Percentage of test subjects showing improvement in skin roughness after application of eye cream of Example 2 for 28 (D28) or 56 (D56) days.

FIG. 11: Skin density measured before treatment (D0), and after 28 (D28) and 56 (D56) days of application of eye cream of Example 2.

FIG. 12: Percentage of subjects showing improved skin density after 28 (D28) and 56 (D56) days of application of the eye cream of Example 2.

FIG. 13: Average firmness values measured before (D0) and after 28 (D28) or 56 (D56) days of application of the eye cream of Example 2.

FIG. 14: Ultrascan images of representative skin samples taken prior to (D0), and after 28 (D28) and 56 (D56) days of application of the eye cream composition of Example 2.

FIG. 15: High resolution images showing improved skin appearance, taken prior to (D0), and after 28 (D28) and 56 (D56) days of application of the eye cream composition of Example 2.

FIG. 16: Wrinkle depth images showing reduced depth of wrinkles in a representative subject, taken prior to (D0), and after 28 (D28) and 56 (D56) days of application of the eye cream composition of Example 2.

FIG. 17: Subjective perceptions of subjects after use of eye cream composition of Example 2 at 28 (D28) and 56 (D56) days.

FIG. 18: Average skin roughness after treatment with the overnight moisturizer composition of Example 3, prior to (D0), and after 28 (D28) and 56 (D56) days of application.

FIG. 19: Percentage of test subjects showing improvement in skin roughness after application of overnight moisturizer composition of Example 3 for 28 (D28) or 56 (D56) days.

FIG. 20: Average melanin values of test subjects prior to (D0), and after 28 (D28) and 56 (D56) days of application.

FIG. 21: Ultrascan images of representative skin samples taken prior to (D0), and after 28 (D28) and 56 (D56) days of application of the overnight moisturizer composition of Example 3.

FIG. 22: High resolution images showing improved skin appearance, taken prior to (D0), and after 28 (D28) and 56 (D56) days of application of the overnight moisturizer composition of Example 3.

FIG. 23: Wrinkle depth images showing reduced depth of wrinkles in a representative subject, taken prior to (D0), and after 28 (D28) and 56 (D56) days of application of the overnight moisturizer composition of Example 3.

FIG. 24: Subjective perceptions of subjects after use of overnight moisturizer composition of Example 3 at 28 (D28) and 56 (D56) days.

FIG. 25: Images of punch biopsy of polyphenol-stained skin treated with glucosyl pentagallate solution (in dimethyl isosorbide), applied 24 days, compared to untreated skin of the same subject. Left image is skin treated with glucosyl pentagallate solution; right image is untreated skin.

FIG. 26: Images of punch biopsy of VVG stained skin treated with glucosyl pentagallate solution (in dimethyl isosorbide), applied 24 days, compared to untreated skin of the same subject. Left image is skin treated with glucosyl pentagallate solution; right image is untreated skin. Elastin appears as black fibers in image.

### DETAILED DESCRIPTION

Human skin comprises three layers of tissue. The outer later, called the epidermis, provides a barrier to environmental pathogens, regulates the amount of water released from the body, and provides mechanical resistance to minor injuries. The dermis layer is the thickest layer of the skin and is disposed below the epidermis. The dermis primarily consists of dense irregular connective tissue and cushions the body from stress and strain. The structural components of the dermis include collagen, elastic fibers composed of elastin, and extrafibrillar matrix. Elastin a key protein constituent of the connective tissue of the skin that affects skin elasticity. Aging and environmental processes can degrade the elastin component of the dermis, contributing to the loss of elasticity and firmness, as well as the formation of wrinkles as skin ages.

In various embodiments, the compositions of the present disclosure are topical compositions comprising glucosyl pentagallate. When suitably formulated to permit effective amounts of glucosyl pentagallate to penetrate through the epidermis and into the dermis layer of the skin, the glucosyl pentagallate can bind to elastin, thereby stabilizing the elastin and reducing degradation. It also allows cell-secreted tropoelastin to anchor and retain in the skin. As a result, the elasticity and firmness of the skin is improved.

Suitable amounts of glucosyl pentagallate range from about 0.1 wt. % to about 2 wt. %, including about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.2 wt. %, about 1.4 wt. %, about 1.6 wt. %, about 1.8 wt. %, or about 2.0 wt. %, inclusive of all ranges between any of these values. In particular embodiments, the amount of glucosyl pentagallate is about 0.5 wt. %.

Evaluations of glucosyl pentagallate topical compositions have shown in human skin fibroblast studies, and in human volunteer studies to increase skin elastin. Animal in vivo studies have shown decreases in skin inflammation, and cell culture studies have demonstrated reduction or prevention of free radical damage.

Gallotannins other than glucosyl pentagallate can also be used in the compositions of the present invention, for example digalloyl glucoses such as 1,6-digalloyl glucose, 2,6-digalloyl glucose, and 3,6-digalloyl glucose; trigalloyl glucoses such as 1,2,3-trigalloyl glucose, 1,2,6-trigalloyl glucose, and 1,3,6-trigalloyl glucose; tetragalloyl glucoses such as 1,2,3,6 tetragalloyl glucose and 1,2,4,6 tetragalloyl glucose; hexagalloyl glucoses, heptagalloyl glucoses, octagalloyl glucoses, nonagalloyl glucoses, and decagalloyl glucoses. Other polyphenols compounds are also suitable for the compositions of the present disclosure, particularly polyphenols containing at least four phenolic groups. A non-limiting list of classes of such compounds includes flavonoids such as anthoxantins (e.g., flavonols, isoflavones, flavones, flavanones, and flavanols), non-flavonoids, phenolic acids, and anthocyanins. Particular compounds include resveratrol, lignans, quercetin, daidzein, apigenin, naringenin, catechin, gallic acid, combinations thereof, and derivatives thereof.

Incorporating glucosyl pentagallate or other polyphenols as described herein into the compositions of the present disclosure, particularly creams or emulsions, can be difficult as glucosyl pentagallate (and other polyphenols) is not higher soluble in water or conventional cosmetic excipients. Accordingly, it must be dissolved in a cosmetically acceptable solvent. Such cosmetically acceptable solvents can include dimethyl isosorbide, dimethyl sulfoxide (DMSO), dimethylacetamide, ethanol, propanol, and fatty alcohols such as cetyl alcohol, stearyl alcohol, cetearyl alcohol, lanoline alcohol, and combinations thereof.

In other embodiments, the compositions of the present disclosure further comprise one of more low molecular weight hyaluronic acids (HAs). Hyaluronic acid is a hydrophilic, anionic, nonsulfated glycosaminoglycan distributed widely throughout connective tissues. The production of HA in the skin decreases with age, and reductions in HA levels in the skin results in lower skin moisture levels, resulting in thinner and more wrinkled appearing skin. In various embodiments, the compositions of the present disclosure comprise two forms of HA, “MicroHA”, and “MiniHA”. MicroHA is hydrolyzed sodium hyaluronate formed as described in EP 3730623 (herein incorporated by reference in its entirety for all purposes) by fermentation of Lactobacillus plantarum, with the accession number of CGMCC No. 16836, to provide low molecular weight hyaluronic acid with a molecular weight of about 1-60 kDa, more particularly less than 5000 Da. MicroHA is available from Bloomage BioTech. MiniHA is oligomeric sodium hyaluronate having a molecular weight of less than 10 kDA, and is also available from Bloomage BioTech. In alternative embodiments, the low molecular weight hyaluronic acid(s) of the present disclosure have a molecular weight capable of penetrating the skin. Typically, molecular weights of more than about 1 kDa do not readily penetrate the skin, so in particular embodiments, the hyaluronic acids of the present invention comprise at least a portion of molecular weights below about 1 kDa. It is understood in the art that hyaluronic acid is typically characterized by the average molecular weight, or by a molecular weight distribution (for example characterized by its polydispersity or some other parameter defining the molecular weight distribution). Accordingly, even if the average molecular weight of the hyaluronic acid is higher than about 1 kDa, the portion of the hyaluronic acid molecular weight distribution below 1 kDa would penetrate the skin, while the portion above 1 kDa would provide a good skin feel to the surface of the skin. Thus, even hyaluronic acids with an average molecular weight above 1 kDa can be suitable for the compositions described herein.

Without being bound by theory, the combination of low molecular weight hyaluronic acid and a polyphenol such as glucosyl pentagallate is effective in increasing the amount of elastin in the skin. The low molecular weight hyaluronic acid increases the amount of tropoelastin (a soluble precursor of elastin) produced by skin cells. By itself, the increased production of tropoelastin produced by application of low molecular weight hyaluronic acid to the skin does not result in higher levels of elastin, as the tropoelastin does not remain in place, and is ultimately eliminated, particularly in older skin which has reduced levels of glycoproteins such as fibrillins. When low molecular weight hyaluronic acid is combined with a polyphenol (such as glucosyl pentagallate), the polyphenol (e.g., glucosyl pentagallate) “anchors” the tropoelastin in place, so that they can crosslink and form elastin fibers in the skin.

Based on cell culture studies, MicroHA inhibits the release of inflammatory cytokines (TNF-alpha, IL-1 alpha, and IL-6) from UV treated keratinocytes, scavenges free radicals, repairs UV damage in fibroblasts and keratinocytes, promotes keratinocyte proliferation, and enhances the barrier function of the skin.

MiniHA is readily absorbed in the skin, and has been shown in in vitro skin testing to penetrate at levels of 36.2%, 60.7%, and 69.5%, 1 h, 8 h, and 24 h, respectively, after application. MiniHA has been shown, like MicroHA, to provide deep moisturizing, promote the repair of damaged cells, scavenge free-radicals, improve skin elasticity, and reduce wrinkle depth.

The amount of MicroHA ranges from about 0.01 to about 0.1 wt. %, including about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, or about 0.1 wt. %, including all ranges between any of these values. In particular embodiments, the amount of MicroHA is about 0.05 wt. %.

The amount of MiniHA in the compositions of the present disclosure ranges from about 0.05 to about 0.15 wt. %, including about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, or about 0.15 wt. %, including all ranges between any of these values. In particular embodiments, the amount of MiniHA is about 0.1 wt. %.

In particular embodiments, the compositions of the present disclosure comprise about 0.1 to about 1 wt. % glucosyl pentagallate and about 0.01 to about 0.15 wt. % of one or more hyaluronic acids independently having a molecular weight of less than about 10 kDa. In more particular embodiments, the compositions of the present disclosure comprise about 0.1 to about 1 wt. % glucosyl pentagallate, about 0.01 to about 0.1 wt. % MicroHA, and about 0.05 to about 0.15 wt. % MiniHA. In yet more particular embodiments, the compositions of the present disclosure comprise about 0.5 wt. % glucosyl pentagallate, about 0.05 wt. % MicroHA, and about 0.1 wt. % MiniHA.

The compositions of the present disclosure, in various embodiments, further comprise peptides such as Argeriline-peptide solution C) (acetyl hexapeptide-3, acetyl hexapeptide-8. Argeriline-peptide solution C has the effect of reducing or preventing/slowing the formation of wrinkles. It is thought to do this by reducing the release of catecholamines (adrenaline and noradrenaline); the over production of such catecholamines is associated with the formation of fine lines and wrinkles. Clinical studies have shown wrinkles around the eyes decreased by up to 10% after 15 days of application, and up to 27% after 30 days of application.

The compositions of the present disclosure, in various embodiments, further comprise Aquaxyl (INCI name: Xylitylglucoside-Anhydroxylitol-Xylitol). Aquaxyl is a complex of natural sugar derivatives. It helps dehydrated skin by acting on three key mechanisms of natural hydration: reinforcing the skin barrier by stimulating ceramide synthesis, optimizing water reserves by boosting natural moisturizing factors in the skin and hyaluronic acid levels; and acting on aquaporins to increase water levels in the epidermis. When present, the amount of Aquaxyl ranges from about 0.1 to about 1 wt. %, including about 0.1, about 0.2, about 0.3, about 0.4, about 0.5, about 0.6, about 0.7, about 0.8, about 0.9, or about 1 wt. %, including all ranges between any of these values. In particular embodiments, the amount of Aquaxyl is about 0.6 wt. %.

The compositions of the present disclosure, in various embodiments, further comprise Ectoin. Ectoin is an amino acid derivative obtained by fermentation. Ectoin is a long-term moisturizer, it repairs UV damage to skin cells, promotes production of collagen and resists aging by improving stress-resistance of skin cells.

The compositions of the present disclosure, in various embodiments, further comprise Equibiome (INCI name: Propylene glycol-Aqua/water-Arctium Lappa Root Extract). Equibiome is obtained from the roots of the Great Burdock plant. It improves the overall skin barrier by increasing cohesion and resistance of the epidermis and reduces excessive pro-inflammatory responses to skin microbes.

The compositions of the present disclosure, in various embodiments, further comprise Matrixyl 3000 (Palmitoyl Oligopeptide, Palmitoyl tetrapeptide-3). Matrixyl 3000 is commercially available from Sederma. Matrixyl 3000 functions synergistically with other components of the compositions of the present disclosure, and has been shown to enhance collagen and elastin production.

Various classes of peptides, such as peptides which are enzyme inhibitors, signal peptides, structural peptides, and neurotransmitter-inhibitor peptides can also be used in the compositions of the present disclosure. Examples of signal peptides include copper tripeptide-1 (also referred to as GHK-Cu, or lamin®), Biopeptide-CL (Pal-GHK), palmitoyl tripeptide-3/5, peptamide-6 (FVAPFP or phe-val-ala-pro-phe-pro), acetyl tetrapeptide-9 (AcTP1), acetyl tetrapeptide-11 (AcTP2), Pal-KTTKS (Palmitoyl pentapeptide-4 or palmitoyl pentapeptide-3 or palmitoyl oligopeptide or Matrixyl®), Tripeptide-10 Citrulline (Decorin-like tetrapeptide or Decorinyl™), human growth hormone, TGF-α, TGF-β, IFN-α, Hsp70, and Aquaporin. Examples of neurotransmitter inhibitor peptides include Acetyl hexapeptide-3 (Argireline® or acetyl hexapeptide-8), Pentapeptide-18 (Leuphasyl®), Pentapeptide-3 (Vialox®), and Syn®-Ake (Tripeptide-3 or dipeptide diaminobutyroyl benzylamide diacetate). Enzyme inhibitor peptides suitable for use in compositions of the present disclosure include Soybean protein/amino acids (Glycine Soja Protein or Preregen®). Examples of structural proteins suitable for use on the compositions of the present disclosure include Keratin proteins/amino acids (Keramino 25®) and Silk protein.

The compositions of the present disclosure, in various embodiments, further comprise Novemer EC-2 Polymer. Novemer EC-2 Polymer is a multifunctional emulsifier. It can be used in both hot and cold manufacturing process, and provides an elegant sensory experience (spreading, cascading effect, nice after-feel).

The compositions of the present disclosure, in various embodiments, further comprise Phytocream 2000: (INCI name: Potassium Palmitoyl Hydrolyzed Wheat protein, Glyceryl Stearate, and Cetearyl alcohol). Phytocream 2000 is a non-ionic emulsifier, particularly for oil-in-water emulsions. Its lipid fraction is compatible with the stratum corneum, and helps restore the structural balance of the natural lipid film, which enhances the skin's protective barrier.

The compositions of the present disclosure, in various embodiments, further comprise Potassium Cetyl Phosphate (e.g., Colafax CPE-K). Potassium Cetyl Phosphate is a water-soluble emulsifier designed to be used as a co-emulsifier, surfactant and emulsifying agent.

In still other embodiments, the compositions of the present disclosure further comprise collagen production promoting amino acids such as γ-aminobutyric acid (GABA), lysine, glycine, and proline. GABA has been shown to reduce wrinkle depth in skin, promote the proliferation of both fibroblasts and keratinocytes, increase the synthesis of collagen and hyaluronic acid in the skin, and improve skin elasticity at levels of up to 10%.

Other additives that promote the formation of collagen include retinoic acid and vitamin C (including ascorbic acid and salts and/or derivatives thereof such as magnesium 1-ascorbic acid 2-phosphate, sodium 1-ascorbic acid 2-phosphate, 1-ascorbic acid 2-glucoside and 1-ascorbic acid ethyl ester

The amount of GABA in the compositions of the present disclosure ranges from about 0.01 to about 0.1 wt. %, including about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, or about 0.1 wt. %, including all ranges between any of these values. In particular embodiments, the amount of GABA is about 0.05 wt. %.

In particular embodiments, the compositions of the present disclosure comprise about 0.1 to about 1 wt. % glucosyl pentagallate, about 0.01 to about 0.15 wt. % of one or more hyaluronic acids independently having a molecular weight of less than about 10 kDa, and about 0.01 to about 0.1 wt. % of a collagen production promoting amino acid such as GABA. In more particular embodiments, the compositions of the present disclosure comprise about 0.1 to about 1 wt. % glucosyl pentagallate, about 0.01 to about 0.1 wt. % MicroHA, about 0.05 to about 0.15 wt. % MiniHA, and about 0.01 to about 0.1 wt. % GABA. In yet more particular embodiments, the compositions of the present disclosure comprise about 0.5 wt. % glucosyl pentagallate, about 0.05 wt. % MicroHA, about 0.1 wt. % MiniHA, and about 0.05 wt. % GABA.

In yet other embodiments, the compositions of the present disclosure further comprise ectoin. Ectoin is 1,4,5,6-tetrahydro-2-methyl-4-pyrimidinecarboxylic acid, and is a natural compound found in several species of bacteria. Ectoin acts as an osmolyte and can protect cells from osmotic stress. In in vitro testing with human cells, Ectoin has been shown to prevent cell damage from UV and visible light, protects against cell stress, inhibits the release if inflammatory factors, and promotes the synthesis of collagen.

When present, the amount of Ectoin in the compositions of the present disclosure ranges from about 0.05 to about 0.15 wt. %, including about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, or about 0.15 wt. %, including all ranges between any of these values. In particular embodiments, the amount of Ectoin is about 0.1 wt. %.

In particular embodiments, for example compositions for the face and neck, the compositions of the present disclosure further comprise Aquaxyl, which is a mixture of xylitylglucoside, anhydroxylitol, and xylitol. Aquaxyl reduces water loss in the skin, and increases the moisturizing efficiency of glycerin.

When present, the amount of Aquaxyl ranges from about 0.1 wt. % to about 1.1 wt. %, including about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, or about 1.2 wt. %, including all ranges between any of these values. In particular embodiments, the amount of Aquaxyl is about 0.6 wt. %.

In particular embodiments, for example compositions for the face and neck, the compositions of the present disclosure further comprise Equibiome, which is a mixture of water, propylene glycol, and Arctium lappa root extract. Equibiome has been shown to reduce or prevent biofilm for S. aureus, increases the barrier of skin to bacteria, and increases moisturization.

When present, the amount of Equibiome ranges from about 0.1 wt. % to about 1.1 wt. %, including about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, or about 1.2 wt. %, including all ranges between any of these values. In particular embodiments, the amount of Equibiome is about 0.6 wt. %.

In particular embodiments, for example compositions for the face and neck, the compositions of the present disclosure further comprise Fluidpure, which is a mixture of hexylene glycol, capryloyl glycine, and xylitylglucoside. Fluidpure reduced irritation and can act as a deodorant by reducing odors.

When present, the amount of Fluidpure ranges from about 0.3 wt. % to about 1.3 wt. %, including about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, or about 1.3 wt. %, including all ranges between any of these values. In particular embodiments, the amount of Fluidpure is about 0.8 wt. %.

In particular embodiments, for example compositions for the face and neck, the compositions of the present disclosure further comprise Matrixyl 3000, which is a mixture of water, pentylene glycol, caprylyl glycol, N-propyl palmitoyl tripeptides. Matrixyl 3000 has been shown to increase collagen production in in vitro cell cultures, and to reduce wrinkles.

When present, the amount of Matrixyl 3000 ranges from about 2 wt. % to about 4 wt. %, including about 2.0 wt. %, about 2.2 wt. %, about 2.4 wt. %, about 2.6 wt. %, about 2.8 wt. %, about 3.0 wt. %, about 3.2 wt. %, about 3.4 wt. %, about 3.6 wt. %, about 3.8 wt. %, or about 4.0 wt. %, including all ranges between any of these values. In particular embodiments, the amount of Matrixyl 3000 is about 2.0 wt. %.

In particular embodiments, for example compositions for overnight moisturizing, the compositions of the present disclosure further comprise Argiriline Peptide Solution C, which is a mixture of acetyl hexapeptide-8 and caprylyl glycol. Argiriline Peptide Solution C has been shown relax muscle contractions, inhibit glutamate release which slows wrinkle activity, and reduces wrinkle volume, length, and depth.

When present, the amount of Argiriline Peptide Solution C ranges from about 2.5 wt. % to about 7.5 wt. %, including about 2.5 wt. %, about 2.7 wt. %, about 2.9 wt. %, about 3.1 wt. %, about 3.3 wt. %, about 3.5 wt. %, about 3.7 wt. %, about 3.9 wt. %, about 4.1 wt. %, about 4.3 wt. %, about 4.5 wt. %, about 4.7 wt. %, about 4.9 wt. %, about 5.1 wt. %, about 5.3 wt. %, about 5.5 wt. %, about 5.7 wt. %, about 5.9 wt. %, about 6.1 wt. %, about 6.3 wt. %, about 6.5 wt. %, about 6.7 wt. %, about 6.9 wt. %, about 7.1 wt. %, about 7.3 wt. %, or about 7.5 wt. %, including all ranges between any of these values. In particular embodiments, the amount of Argiriline Peptide Solution C is about 5.0 wt. %.

In particular embodiments, for example compositions of the present disclosure formulated as eye creams, the compositions further comprise “Eyeliss”, which is a mixture of water, glycerin, hesperidin methyl chalcone, steareth-20, dipeptide-2, and palmitoyl tetrapeptide-7. Eyeliss has been shown to improve lymphatic circulation, improve capillary fragility, and is effective in reducing puffiness of the skin around the eyes, and reducing bags under the eyes.

In such compositions, the amount of Eyeliss ranges from about 1.5 wt. % to about 2.5 wt. %, including about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, or about 2.5 wt. %, including all ranges between any of these values.

In particular embodiments, the compositions of the present disclosure comprise glucosyl pentagallate, hydrolyzed sodium hyaluronate, and aminobutyric acid. In particular embodiments, the compositions of the present disclosure comprise glucosyl pentagallate, hydrolyzed sodium hyaluronate, and aminobutyric acid, wherein the hydrolyzed sodium hyaluronate is a mixture of at least two different grades of hydrolyzed sodium hyaluronate, e.g., one having a molecular weight ranging from about 1-60 kDa, and the other having a molecular weight of less than 10 kDa. In more particular embodiments, the hydrolyzed sodium hyaluronate polymers comprise a mixture of Micro HA and Mini HA as described herein.

The compositions of the present disclosure, in various embodiments, further comprise additional cosmetically acceptable ingredients selected from the group consisting of fragrances, dyes, colorants, adsorbents, lubricants, solvents, moisturizers (emollients, humectants, film-formers, occlusive agents), water repellants, UV absorbers (physical absorbers such as titanium dioxide, zinc oxide, etc.; chemical absorbers such as para-aminobenzoic acid (PABA) and PABA derivatives), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and mannitol), exfoliants, waterproofing agents (e.g., magnesium/aluminum hydroxide stearate), and skin conditioning agents (e.g., aloe extracts, allantoin, bisabolol, ceramides, dimethicone, hyaluronic acid, and dipotassium glycyrrhizate).

Cosmetically suitable fragrances suitable for use in the compositions of the present disclosure include, for example, natural or synthetic odoriferous substances, or mixtures thereof, which individually or in combination generate an attractive scent. Non-limiting examples of natural odoriferous substances include extracts of flowers (lily, lavender, rose, jasmine, neroli or ylang-ylang), stems and leaves (geranium, patchouli, petitgrain), fruits (anis, coriander, caraway, juniper), fruit rinds (bergamot, lemon, orange), roots (mace, angelica, celery, cardamom, costus, iris, thyme), needles and twigs (spruce, fir, pine, mountain pine) and resins and balsams (galbanum, elemi, benzoin, myrrh, frankincense, opoponax). Typical synthetic perfume compounds comprise esters, ethers, aldehydes, ketones, alcohols and hydrocarbons. Essential oils of low volatility, which are generally used as flavoring components, are also suitable as fragrances, for example, but not limited to, sage oil, camomile oil, clove oil, balm oil, peppermint oil, cinnamon leaf oil, linden blossom oil, juniper berry oil, vetiver oil, frankincense oil, galbanum oil, labdanum oil and lavandin oil.

A non-limiting list of cosmetically suitable moisturizers include petrolatum, lanolin, mineral oil, dimethicone (silicone fluid), cetyl alcohol, cetearyl alcohol, cocoa butter, isopropyl myristate, isopropyl palmitate, isopropyl isostearate, diisopropyl adipate, diisopropyl sebacate, lanolin, liquid paraffin, shea butter, silicone oils, stearic acid, stearyl alcohol, castor oil, glycerin, lecithin, propylene glycol, and ethylene glycol.

A non-limiting list of cosmetically acceptable emulsifiers includes polyglyeryl-based emulsifiers, polyol esters, glycerol ethers, oxyethylenated and/or oxypropylenated ethers, ethylene glycol polymers, sorbitan esters, nonionic or surfactant-type emulsifiers, e.g., alkanolamides; alkyl polyglucosides; polyoxyalkylenated nonionic surfactants;

polyglycerolated nonionic surfactants; ethoxylated fatty esters; alcohols, alpha-diols, alkylphenols and esters of fatty acids, being ethoxylated, propoxylated or glycerolated;

copolymers of ethylene oxide and/or of propylene oxide; condensates of ethylene oxide and/or of propylene oxide with fatty alcohols; polyethoxylated fatty amides; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N-(C₆-C₂₄)alkylglucamine derivatives, amine oxides such as (C₁₀-C₁₄)alkylamine oxides or N-(C₁₀-C₁₄)acylaminopropylmorpholine oxides; and mixtures thereof.

The pH of the cosmetic compositions of the present disclosure may be from about 4 to about 12, including 4, 4.5, 5, 5.5, 6, 6.5, 7, 7.5, 8, 8.5, 9, 9.5, 10, 10.5, 11, 11.5, or 12, including any pH range between any of these values. The pH of the compositions of the present disclosure may be adjusted to the desired value using at least one acidifying agent or base that is well-known in the art.

The cosmetic compositions of the present disclosure are generally in the form of emulsions. The emulsions of the present disclosure can be oil-in-water emulsions, water-in-oil emulsions, water-in-oil-in-water emulsions, oil-in-water-in-oil emulsions, and so forth. The compositions of the present disclosure can also comprise suspensions or solutions.

The compositions of the present disclosure, when applied in cosmetically appropriate amounts to the skin, improve the elasticity and firmness of the skin.

The compositions of the present disclosure are applied to the skin, typically daily, but in alternative embodiments, two or more times per day (e.g., morning and evening). The amount applied can range from about 0.5/cm² to about 10 g/cm², including about 0.5, about 0.6, about 0.7, about 0.8, about 0.9, about 1, about 2 about 3, about 4, about 5, about 6, about 7, about 8, about 9, or about 10 g/cm², inclusive of any range between any of these values. Application to skin means depositing an amount of the compositions of the present disclosure to the skin, using e.g., the fingers or an applicator, then smoothing and spreading the composition on the surface of the skin sufficiently to absorb or spread as a thin layer on the skin.

Compositions of the present disclosure that are formulated as an overnight moisturizer include emollients such as fatty acid esters, isopropyl isostearate, Phytocream 2000 (a mixture of potassium palmitoyl hydrolyzed wheat protein, glycerol stearate, and cetearyl alcohol), diisopropyl adipate, diisopropyl sebacate, silicone oils, etc., and film forming agents such as Xpertmoist Molecular Film (INCI name: Water (Aqua), Glycerin, Pseudoalteromonas Ferment Extract, Xanthan Gum, Proline, Alanine, Serine, Sodium Phosphate, Sodium Hydroxide, Tocopherol, Caprylyl Glycol, Ethylhexylglycerin), Actimoist Bio-1PF (INCI name: Water (and) Sodium Hyaluronate), and November EC-2 Polymer (INCI name: Sodium Acrylates/Beheneth-25 Methacrylate Crosspolymer (and) Hydrogenated Polydecene (and) Lauryl Glucoside).

The compositions of the disclosure, when applied to the skin on at least a daily basis, in amounts ranging from 0.5-10 g/cm², provide one or more of improved firmness, skin thickness, elasticity, increased hydration, reduced roughness, or reduced wrinkle depth. The amount of improvement ranges from at least about 5%, to up to about 100% or more, including about 5%, about 10%, about 15%, about 20%, about 25%, about 30%, about 35%, about 40%, about 45%, about 50%, about 55%, about 60%, about 65%, about 70%, about 80%, about 90%, about 95%, or about 100%, including any ranges between any of these values. The improvements can be measured using methods known in the art for measuring changes in skin firmness, skin thickness, elasticity, hydration, roughness, or wrinkle depth, including the methods disclosed herein.

### EXAMPLES

### Example 1: Facial Neck Firming Gel Serum

A facial and neck firming serum having the following composition shown in Table A was prepared:

TABLE A

| | | Wt. |
|---|---|---|
| Phase | Ingredient(s) | % |
| A | One or more dimethicone fluids | 36 |
| B | Water, buffer, NaCl | 20.85 |
| | GABA | 0.05 |
| | Micro HA | 0.05 |
| | MiniHA | 0.5 |
| | Ectoin | 0.1 |
| C | Glucosyl pentagallate | 0.5 |
| | Preservative(s), humectant(s), emollient(s), conditioning | 36.35 |
| | agent(s), solvent(s), odor control agent(s) | |
| D | Plant extract(s), barrier agent(s), skin purifying | 6 |
| | agent(s), moisturizer(s) | |

1. Homogenize phase A at room temperature.

2. Mix phase B under gentle stirring at room temperature.

3. For phase C:

a. Dissolve glucosyl pentagallate in one or more cosmetically acceptable solvents.

b. Separately mix together the remaining phase C ingredients until a homogenous mixture is obtained.

c. Add b to a under constant stirring.

4. Separately mix together the phase D ingredients 18-21 until a homogenous mixture is obtained.

5. Very slowly add phases B, C, and D into phase A while stirring, until a more viscous and homogenous gel is obtained.

6. Fill the batch in a suitable vessel and store the batch at room temperature.

### Example 2: Firming Eye Cream

A firming eye cream having the following composition shown in Table B was prepared:

TABLE B

| | | Wt. |
|---|---|---|
| Phase | Ingredient(s) | % |
| A | Water, chelating agent(s), emollient(s) and/or humectant(s), | 81.23 |
| | polymeric emulsifier(s), skin purifying agent(s), collagen | |
| | stimulating/wrinkle-reducing peptide(s) | |
| | GABA | 0.05 |
| | Micro HA | 0.05 |
| | Mini HA | 0.10 |
| B | Surfactant(s) and/or emulsifier(s), pigment(s) | 0.4 |
| C | Silicone(s), emollient(s) | 13.5 |
| D | pH adjusting agent(s), preservative(s), agent(s) for | 3.12 |
| | reducing fluid retention and fine lines in the skin | |
| E | Glucosyl pentagallate | 0.5 |
| | Solvent(s) | 1.5 |

1. Mix together, with stirring and heating, phase A ingredients to form a homogeneous mixture.

2. One at a time, add phase B ingredients to phase A. Mix until uniform, after each addition.

3. Add phase C ingredients one at a time to the mixture of phase A and B, with heating, and mix until homogeneous.

4. Add phase D ingredients one at a time to mixture of phases A-C, mixing well between each addition.

5. Separately mix together phase E ingredients to form homogenous mixture, then add to mixture of phases A-D, with stirring, to form homogeneous mixture.

### Example 3: Hydrating Overnight Moisturizer

A hydrating overnight moisturizer having the following composition shown in Table C was prepared:

TABLE C

| | | Wt. |
|---|---|---|
| Phase | Ingredient(s) | % |
| A | Water, humectant(s) and/or emollient(s), rheological | 69.5 |
| | modifier(s), surfactant(s), solvent(s), | |
| | GABA | 0.05 |
| | Micro HA | 0.5 |
| | Mini HA | 0.1 |
| | Glucosyl pentagallate | 0.5 |
| B | Emollient(s), moisturizer(s), anti-inflammatory agent(s), | 14 |
| | silicone(s) | |
| C | Moisturizing/barrier film(s), moisturizer(s), peptide(s), | 15.5 |
| | emulsifier/rheology modifier(s) | |
| D | pH control agent(s) | 0.3 |

1. Mix glucosyl pentagallate and solvent(s) to form a homogeneous mixture. Mix remaining ingredients in phase A to form a homogeneous mixture. Slowly add the glucosyl pentagallate solution to the mixture of phase A ingredients to form a homogeneous mixture.

2. In a separate vessel, mix ingredients of phase B with heating until homogenous, then add to phase A mixture.

3. Add ingredients of phase C successively into the mixture of phases A-B. Mix well between each addition.

4. Adjust the pH to about 6.0-6.5 with the pH control agent(s) of phase D.

### Example 4: Insoluble Elastin Production

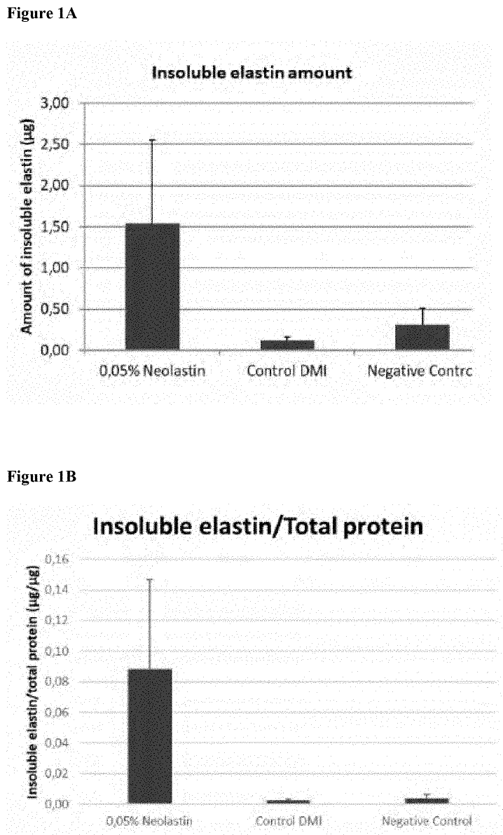

Human dermal fibroblast cells (CCD-1064sk (ATCC® CRL-2076TM)) were cultured at a seeding density of 200,000 cells/well (12-well plate) and incubated in a humidified atmosphere at 37° C. and 5% CO₂ with 0.05% glucosyl pentagallate (Neolastin) dissolved in dimethyl isosorbide (DMI). As a control, cells were also exposed to DMI without glucosyl pentagallate. 4 replicate samples were prepared. The negative control did not include DMI or glucosyl pentagallate. After 14 days of culturing, cells cultured in the presence of 0.05 glucosyl pentagallate showed a significant increase in the production of insoluble elastin. See FIGS. 1A-C. This shows that cosmetic compositions comprising glucosyl pentagallate can stimulate production of insoluble elastin in the skin, thereby improving skin firmness and appearance.

### Example 5: Neck Firming Cream

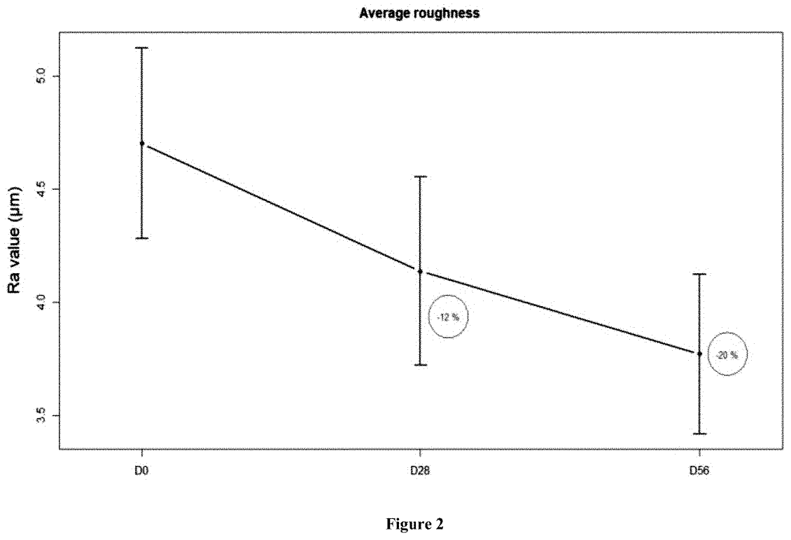

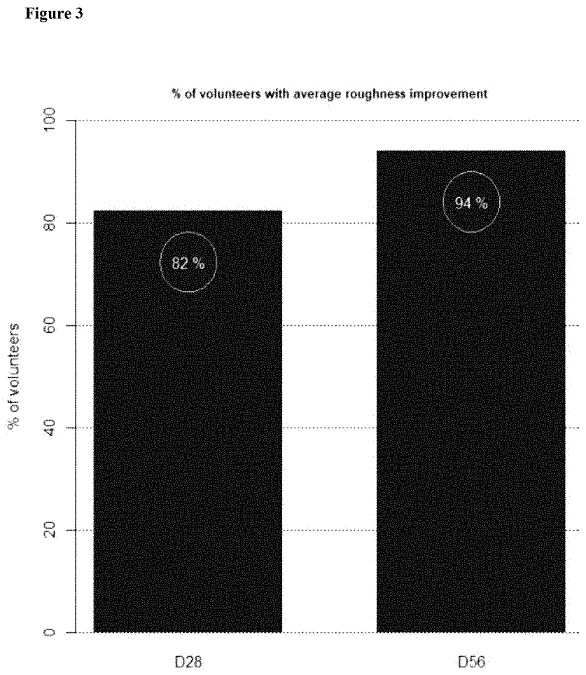

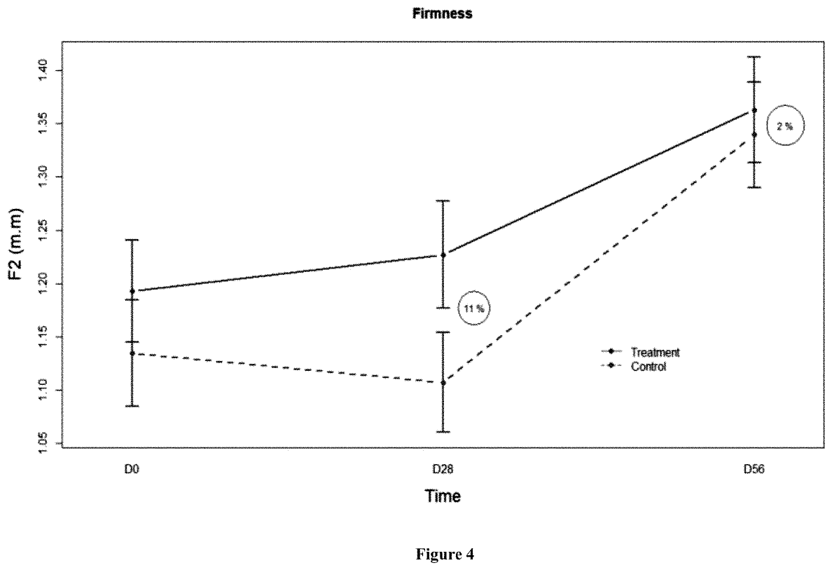

The neck firming cream described in Example 1 was tested on human subjects. After 28 and 56 days of applications, the subjects an average reduction in skin roughness of 12% and 20%, respectively. See FIG. 2, where lower Rₐ values indicate lower roughness. The percentage of subjects reporting improvements in perceived roughness at 28 days was 82%, and at 56 days, 94%. See FIG. 3. After 28 days, the average increase in skin firmness was 11% after 28 days. See FIG. 4, where high F₂ values indicate higher firmness. The average increase in skin hydration after 24 hours and 56 days (long term hydration) was 9%.

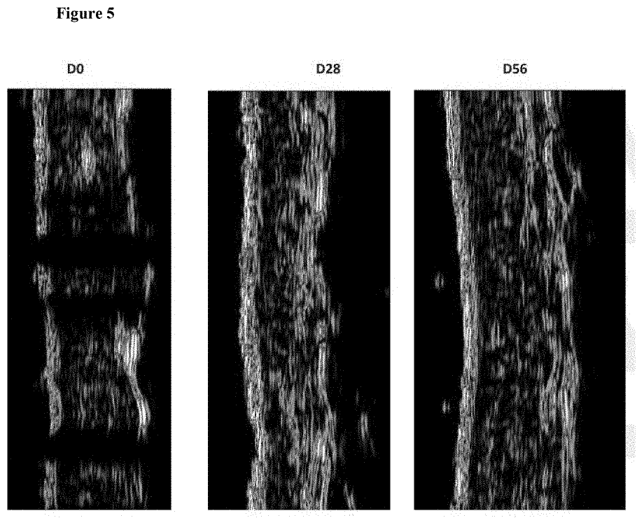

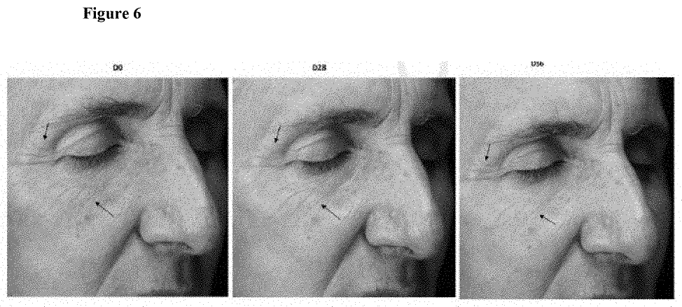

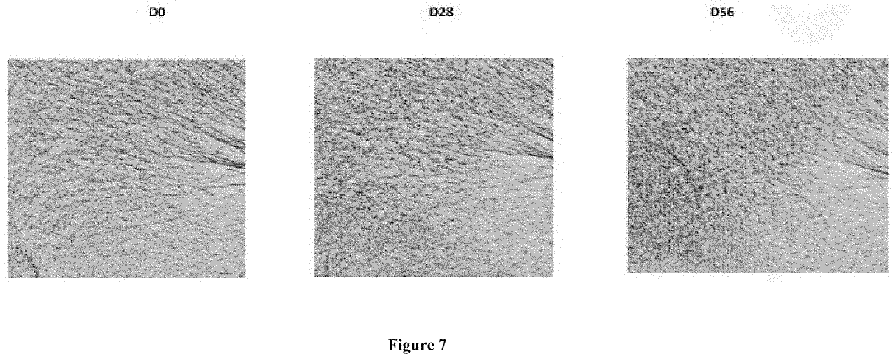

Ultrascan results of subject skin after treatment with neck firming cream, taken initially (D0), at 28 (D28) and 56 (D56) days show improvements in skin structure, as shown in FIG. 5. High-definition photos of wrinkles on a test subject (FIGS. 6 and 7) taken initially (D0), at 28 (D28) and 56 (D56) days show reductions in the appearance of fine lines and wrinkles.

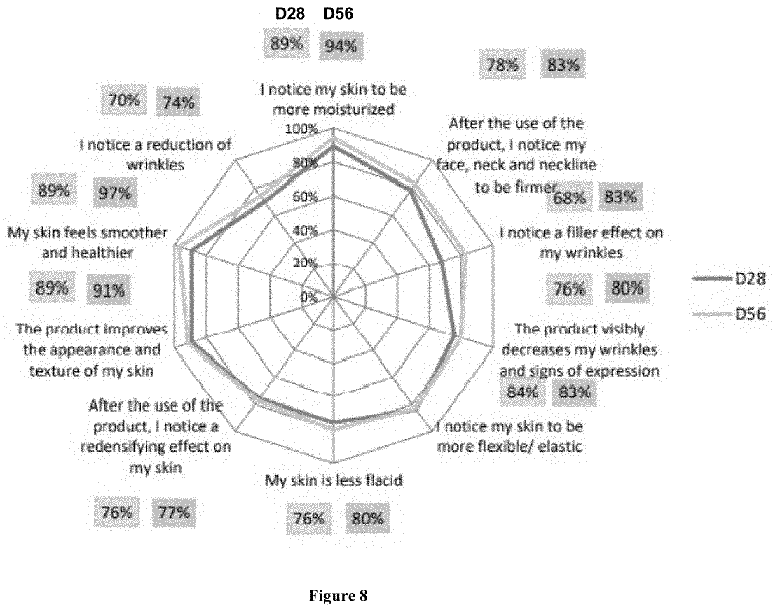

Overall, after 56 days, 97% of the test subjects noticed smoother and healthier skin; 94% felt their skin was more moisturized, smoother, and healthier; 91% felt the neck firming cream improved skin appearance and texture; 83% noticed firmer skin on the face and neckline, and wrinkles appeared to be reduced (filler effect); and 80% noticed less flaccidity in the skin. See FIG. 8.

### Example 6: Eye Cream

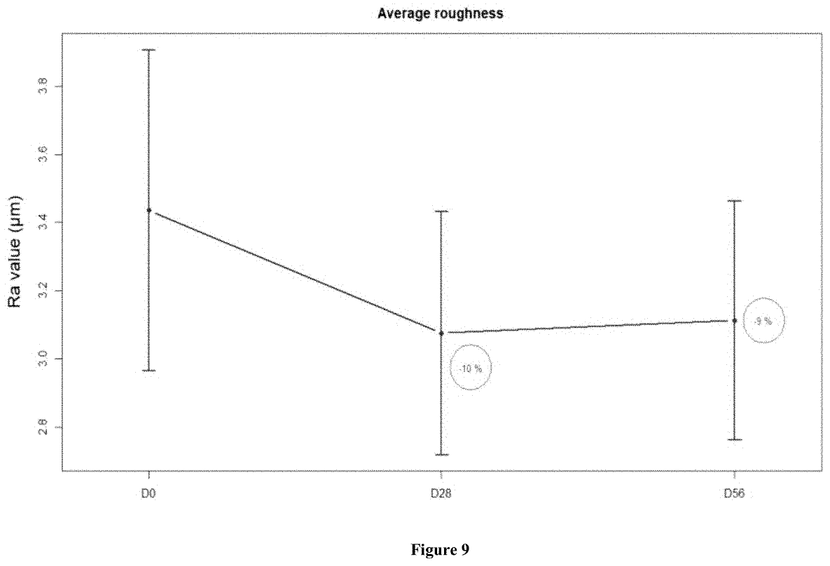

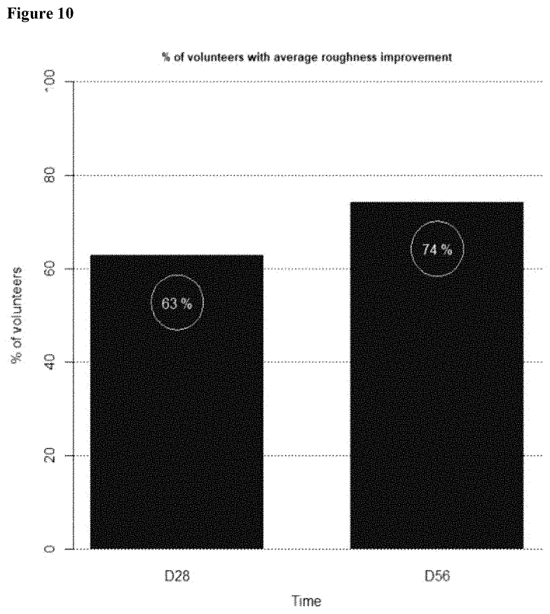

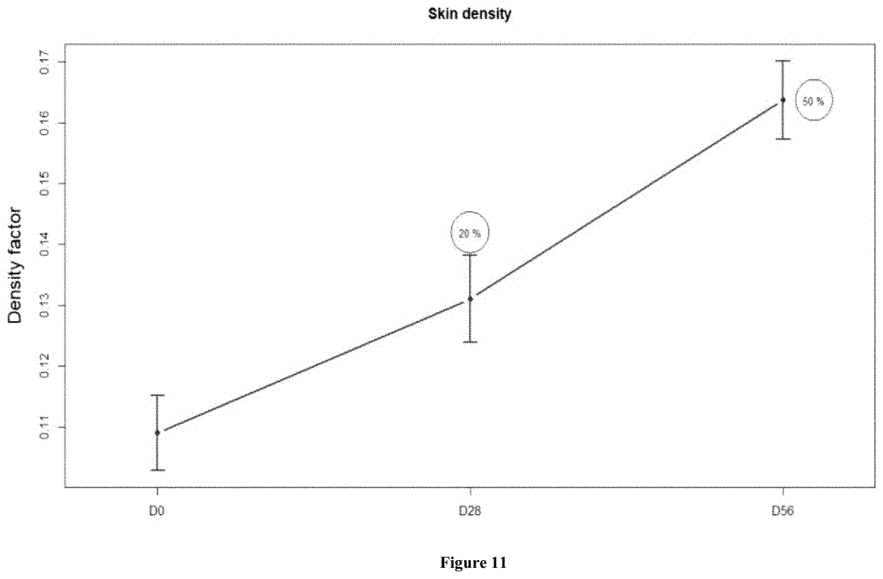

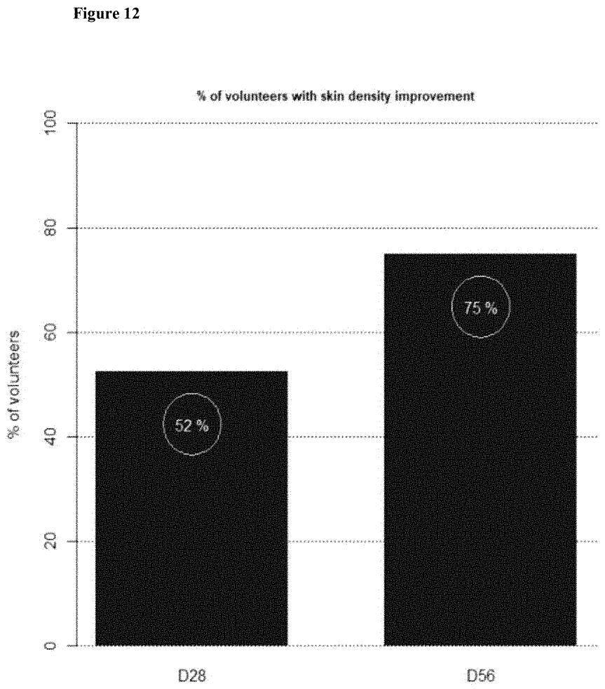

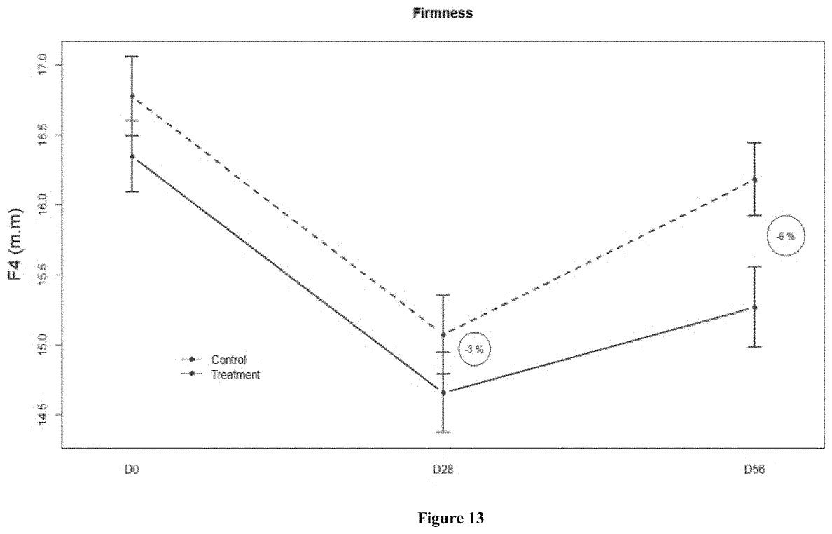

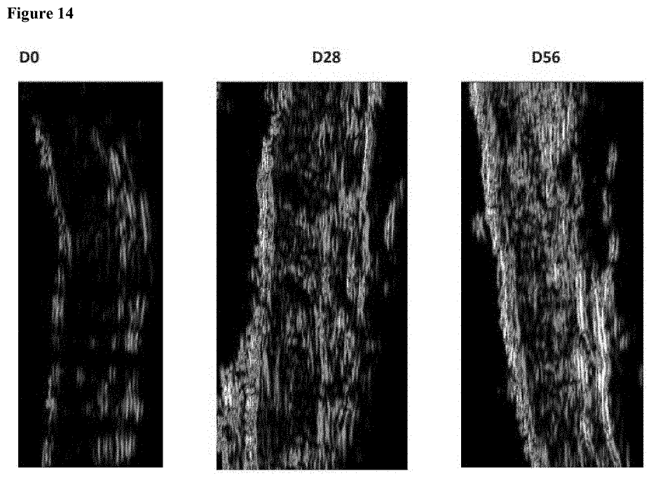

The eye cream described in Example 2 was tested on human subjects. After 28 and 56 days of application, the subjects an average reduction in skin roughness of 10% and 9%, respectively. See FIG. 9, where lower Rₐ values indicate lower roughness. The percentage of subjects reporting improvements in perceived roughness at 28 days was 63%, and at 56 days, 74%. See FIG. 10. The average increase in skin firmness as measured by ultrascan was 20% after 28 days and 50% in 56 days. See FIG. 11. The percentage of subjects at 28 and 56 days who perceived an improvement in skin density was 52% and 75%, respectively, See FIG. 12. Similar improvements in skin firmness were obtained by the alternative cutometer method. Improvements in skin firmness over controls (cosmetic vehicle without glucosyl pentagallate were noted at 28 and 56 days. See FIG. 13, where lower F₄ values indicate high firmness. Improved skin structure was also observed using ultrascan images of a representative test subject, before treatment (D0), and after 28 (D28) or 56 (D56) days of application. See FIG. 14.

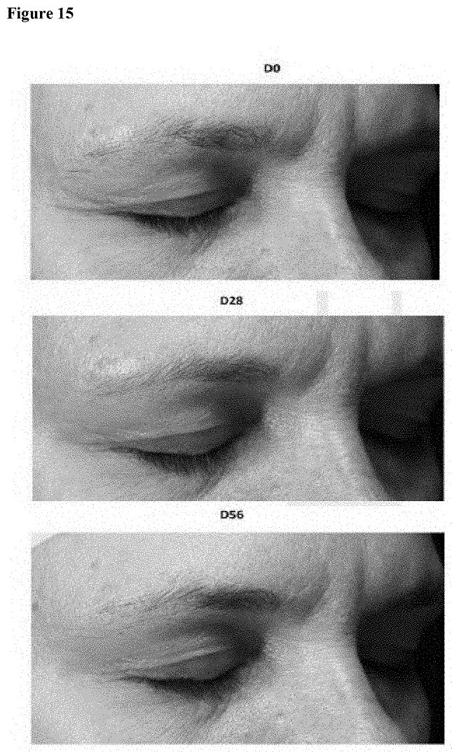

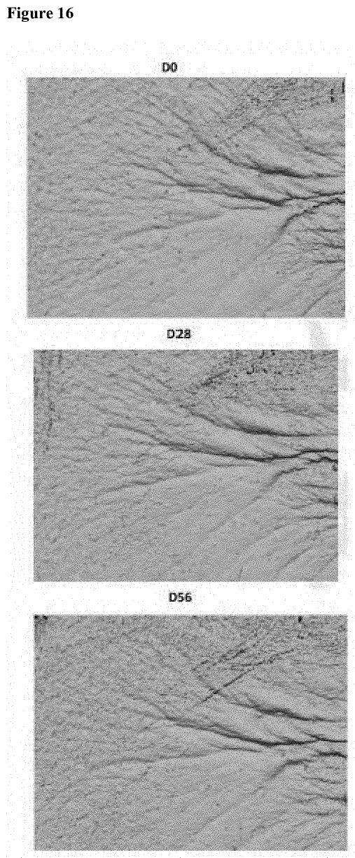

High-definition images of the eye region of a representative test subject also show the reduction in the appearance of fine lines and wrinkles, before treatment (D0), and after 28 (D28) or 56 (D56) days of application. See FIGS. 15-16.

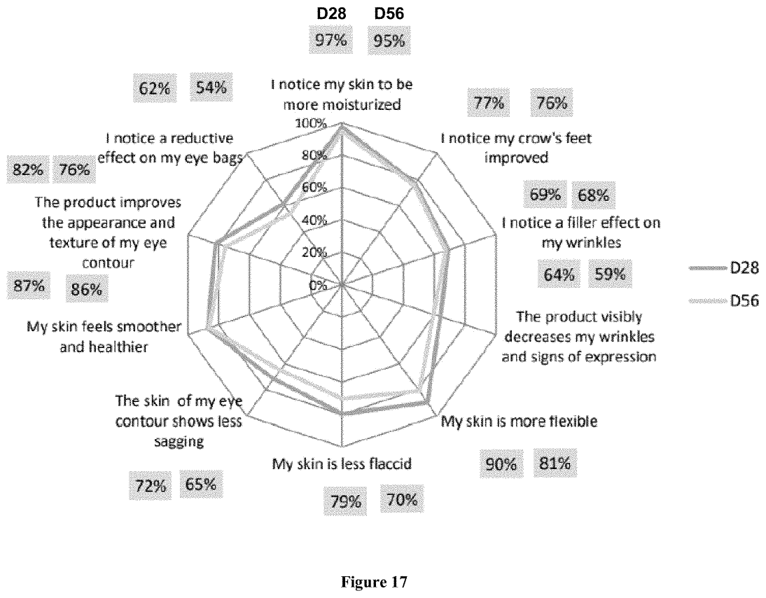

Overall, after 28 days, 97% of the test subjects felt their skin was more moisturized, smoother, and healthier; 90% perceived their skin was more flexible; 87% felt their skin was smoother and healthier; and 82% considered that the eye cream improved the appearance and texture of their skin. See FIG. 17.

### Example 7: Overnight Moisturizer

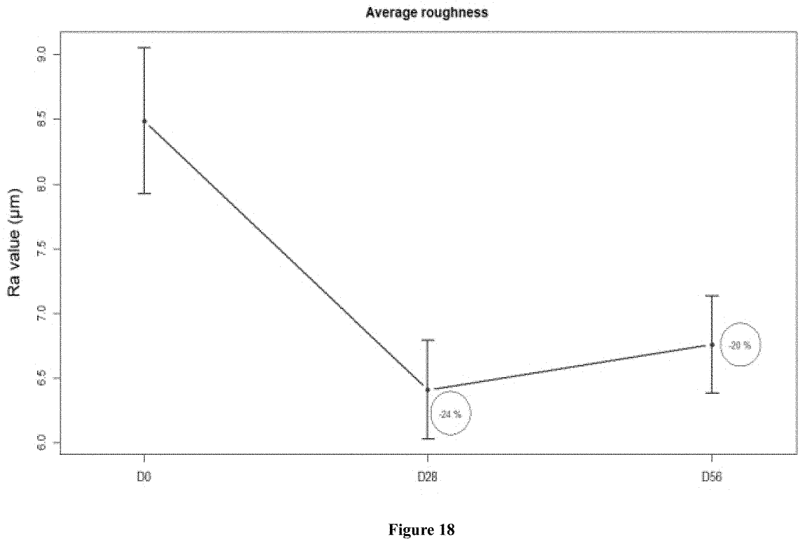

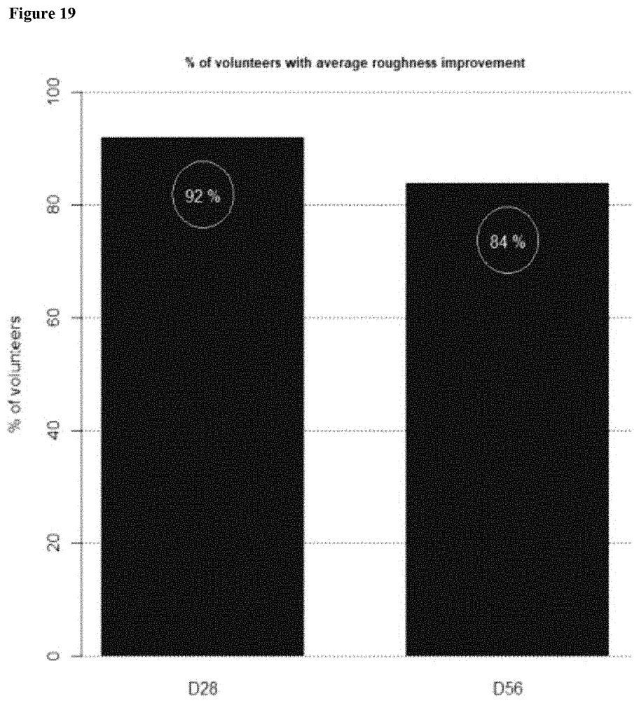

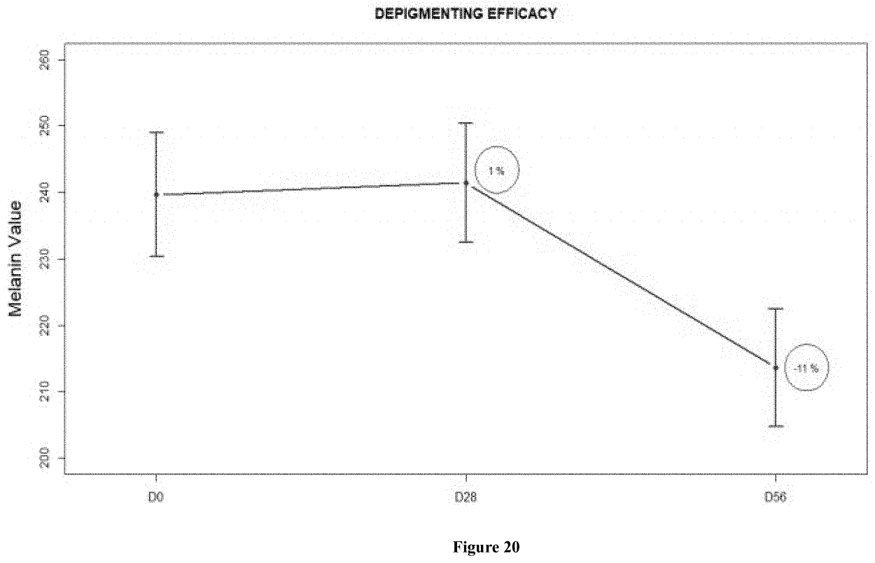

The overnight moisturizer described in Example 3 was tested on human subjects. After 8 and 24 hours after application, the subjects showed an average increase of 6% in skin hydration. After 28 and 56 days of application, the subjects showed an average reduction in skin roughness of 24% and 20%, respectively. See FIG. 18, where lower Rₐ values indicate lower roughness. The percentage of subjects reporting improvements in perceived roughness at 28 days was 92%, and at 56 days, 84%. See FIG. 19. As measured my reductions in melanin, subjects showed an average reduction in pigmentation (i.e., lower melanin values) of 11% after 56 days if use. See FIG. 20.

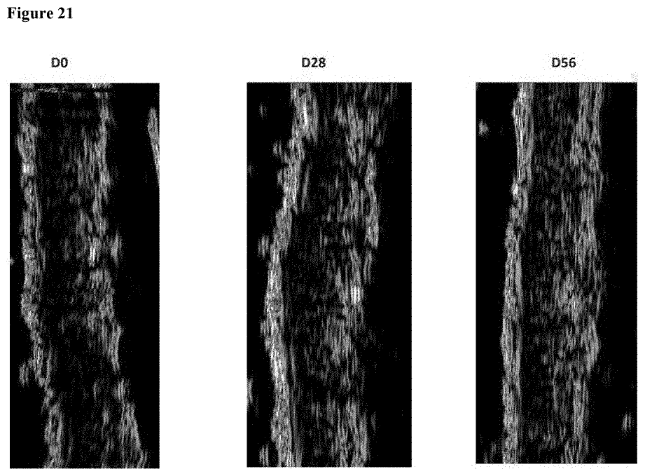

Improved skin structure was observed using ultrascan images of a representative test subject, before treatment (D0), and after 28 (D28) or 56 (D56) days of application. See FIG. 21.

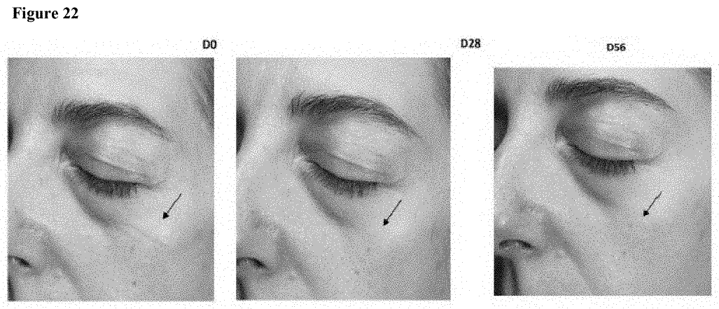

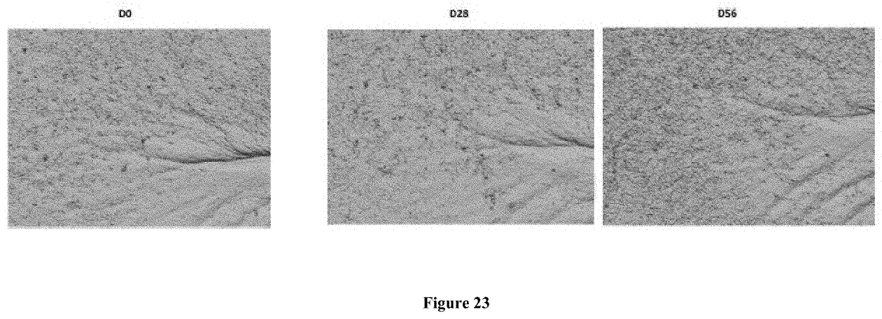

High-definition images of the eye region of a representative test subject also show the reduction in the appearance of fine lines and wrinkles, before treatment (D0), and after 28 (D28) or 56 (D56) days of application. See FIGS. 22-23.

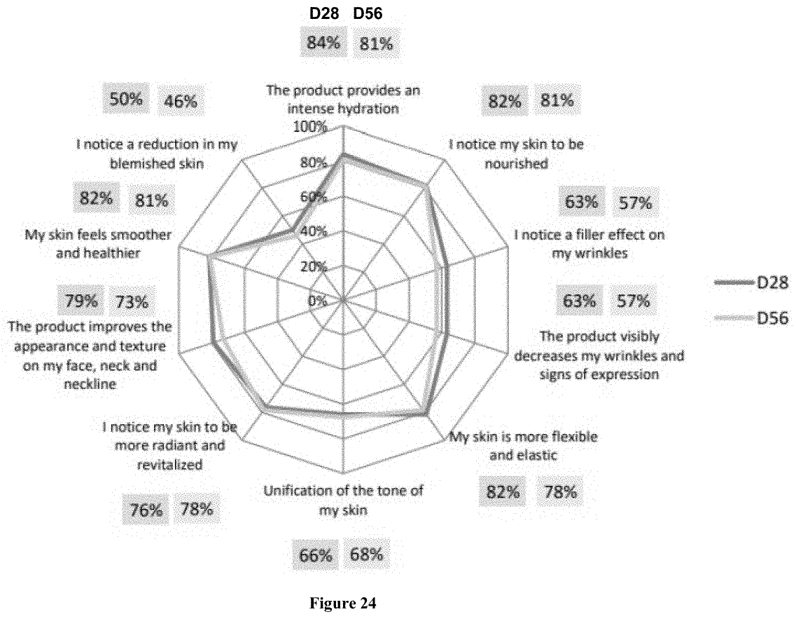

Overall, after 28 days, 84% of the test subjects felt that the overnight moisturizer provided intense skin hydration; 82% noticed smoother and healthier appearing skin; and 82% perceived their skin to be more flexible and elastic. See FIG. 24.

### Example 8: Comparison of Histology of Polyphenol-Stained Punch Biopsy—Treated Vs. Untreated Skin

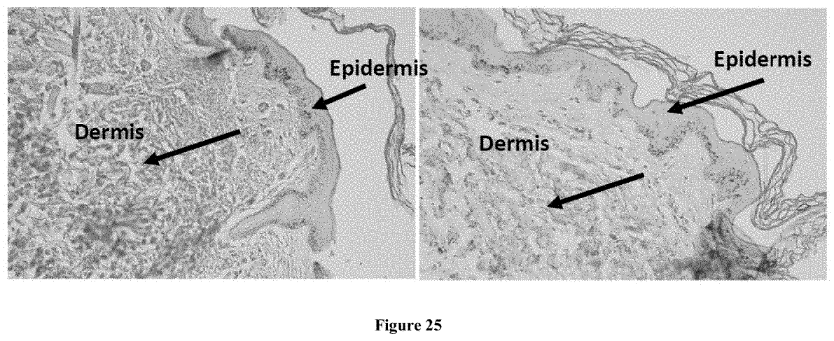

The skin of a subject's left arm 2 cm² patch (upper under arm above elbow) was treated with a 25% dimethyl isosorbide solution of glucosyl pentagallate for 24 days. Biopsy samples of the treated skin and a similar sample of untreated skin from the same subject's right were then taken, sectioned, and stained for polyphenol (Ferric Chloride stain) and imaged with a light microscope. As shown in the left-side image of FIG. 25, treated skin exhibited more black staining in the dermis showing glucosyl pentagallate in the dermis and denser tissue overall, compared to the untreated skin sample (right side image).

### Example 9: Comparison of Histology of Elastin (VVG Stained) Punch Biopsy—Treated Vs. Untreated Skin

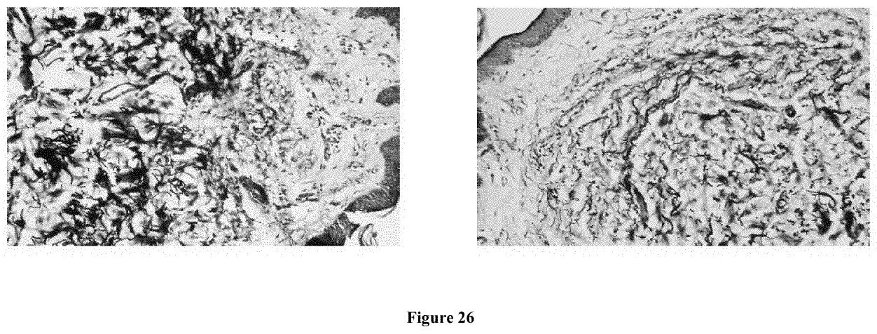

The skin of a subject's left arm was treated with a 25% dimethyl isosorbide solution of glucosyl pentagallate for 24 days. Biopsy samples of the treated skin and a similar sample of untreated skin from the same subject's right were then taken, sectioned and stained for elastic fibers (Verhoeff-Van Gieson, VVG stain that stains elastic fibers black. As shown in the left-side image of FIG. 26, treated skin exhibited significant black staining (indicating higher levels of elastin fibers) compared to the untreated skin sample (right side image).

1. A topical composition comprising:
a) about 0.2-0.6 wt. % glucosyl pentagallate;
b) about 0.02-0.07 wt. % micro HA; and
c) about 0.05-0.15 wt. % mini HA;
and one or more cosmetically acceptable excipients selected from the group consisting of a UV protectant, an emollient, a moisturizer, an antibacterial agent, an emulsifier, and combinations thereof, and c.

2. The topical composition of claim 1, further comprising about 0.02-0.07 wt. % aminobutyric acid.

3. The topical composition of claim 1, wherein after daily administration of 1-10 g/cm² on the epidermis of a subject for at least 28 days, the epidermis has improved firmness, elasticity, increased hydration, reduced roughness, or reduced wrinkle depth.

4. The topical composition of claim 1, in the form of an oil-in-water emulsion.

5. The topical composition of claim 1, comprising:
a) about 0.5 wt. % glucosyl pentagallate;
b) about 0.05 wt. % aminobutyric acid;
c) about 0.05 wt. % micro HA; and
d) about 0.10 wt. % mini HA.

6. A method of reducing wrinkles in the epidermal tissue of a subject, comprising daily administration of the topical composition of claim 1, in an amount of 1-10 g/cm², whereby after administration of said topical composition for at least 28 days, the epidermis has a reduction in wrinkles of at least 5%.

7. A method of increasing elasticity in the epidermal tissue of a subject, comprising daily administration of the topical composition of claim 1, in an amount of 1-10 g/cm², whereby after administration of said topical composition for at least 28 days, the epidermis has an increase in elasticity of at least 5%.

8. A method of increasing firmness in the epidermal tissue of a subject, comprising daily administration of the topical composition of claim 1, in an amount of 1-10 g/cm², whereby after administration of said topical composition for at least 28 days, the epidermis has an increase in firmness of at least 10%.

9. A method of decreasing roughness in the epidermal tissue of a subject, comprising daily administration of the topical composition of claim 1, in an amount of 1-10 g/cm², whereby after administration of said topical composition for at least 28 days, the epidermis has a decrease in roughness of at least 10%.

10. A method of increasing hydration in the epidermal tissue of a subject, comprising daily administration of the topical composition of claim 1, in an amount of 1-10 g/cm², whereby after administration of said topical composition for at least 28 days, the epidermis has an increase in hydration of at least 5%.

11. A method of increasing density of the epidermal tissue of a subject, comprising daily administration of the topical composition of claim 1, in an amount of 1-10 g/cm², whereby after administration of said topical composition for at least 28 days, the epidermis has an increase in skin density of at least 20%.

12. The method of claim 6, wherein the topical composition further comprises about 0.02-0.07 wt. % aminobutyric acid.

13. The method of claim 6, wherein after daily administration of 1-10 g/cm² of the topical composition on the epidermis of a subject for at least 28 days, the epidermis has improved firmness, elasticity, increased hydration, reduced roughness, or reduced wrinkle depth.

14. The method of claim 6, wherein the topical composition is in the form of an oil-in-water emulsion.

15. The method of claim 6, wherein the topical composition comprises:
a) about 0.5 wt. % glucosyl pentagallate;
b) about 0.05 wt. % aminobutyric acid;
c) about 0.05 wt. % micro HA; and
d) about 0.10 wt. % mini HA.
